(19) United States
(12) Patent Application Publication
(10) Pub. No.: US 2020/0405727 A1
(43) Pub. Date: Dec. 31, 2020

(54) NEW ALK INHIBITOR SENOLYTIC DRUGS

(71) Applicant: FONDAZIONE PER L'ISTITUTO ONCOLOGICO DI RICERCA (IOR), Bellinzona (CH)

(72) Inventors: Manuel COLUCCI, Bellinzona (CH); Ajinkya REVANDKAR, Bellinzona (CH); Andrea ALIMONTI, Milano (IT)

(73) Assignee: FONDAZIONE PER L'ISTITUTO ONCOLOGICO DI RICERCA (IOR), Bellinzona (CH)

(21) Appl. No.: 16/962,602

(22) PCT Filed: Jan. 15, 2019
(86) PCT No.: PCT/IB2019/050304

§ 371 Date: Jul. 16, 2020

(30) Foreign Application Priority Data
Jan. 17, 2018 (IT) 102018000001168

(51) Int. Cl. A61K 31/5377; A61P 35/00; A61K 31/337; A61K 31/519; A61K 33/243; A61K 31/704; A61P 17/18

(52) U.S. Cl. CPC A61K 31/5377; A61P 35/00; A61K 31/337; A61P 17/18; A61K 33/243; A61K 31/704; A61K 31/519

(57) ABSTRACT

The present invention relates to the use of ALK tyrosine-kinase receptor inhibitors, in particular Alectinib, and compositions including it, as senolytic drug. In particular it relates to the use as senolytic drugs in a method of treatment of tumour or other pathological and incapacitating conditions associated to the cellular senescence.

The present invention relates to the use of ALK tyrosine-kinase receptor inhibitors, in particular Alectinib, and compositions including it, as senolytic drug. In particular it relates to the use of ALK tyrosine-kinase receptor inhibitors, in particular Alectinib, as senolytic drug in a method of treatment of tumour or other conditions associated to the cellular senescence.

### STATE OF PRIOR ART

Senescence is a state wherein the cell, in response to precise stimuli, loses the replicative capability even if it maintains metabolically active. It carries out physiological important roles during the ageing processes and it results to be crucial in contrasting the uncontrolled growth of the tumour cells. Notwithstanding the senescent cells are in a not proliferative state, they however remain metabolically active and start to secrete a wide number of cytokines and pro-inflammatory factors, thus assuming a secretory phenotype (senescence-associated secretory phenotype, SASP). This “secretome” is capable of activating the antitumour immune response and promoting the removal of the senescent cells themselves (“senescence surveillance”). However, there are cases wherein the senescent cells avoid the protective action of the immune system by remaining in the cancerous site and by carrying out, through the secretome, a protumour action capable of promoting angiogenesis, migration and cellular invasiveness and, consequently, the pro-metastatic capability. In this context then the senolytic therapy, that is the specific removal of the senescent cells, appears as a new clinical strategy apt to improve the effectiveness of the canonical chemotherapies.

Nowadays, the number of senolytic drugs on the market results to be poor and most part thereof is directed against Bcl-2, a known survival regulator typically over-expressed by the senescent cells. The effectiveness of these compounds however is variable and strictly connected with the genetics of the senescent cells themselves, consequently it varies depending upon the different inter and intra-tumour features. Moreover, the senolytic drugs can be used in the prevention and/or treatment of all pathologies associated to the cellular senescence.

The object of the present invention is to provide new substances with senolytic activity for use in the treatment of tumours and other pathologies associated to the cellular senescence.

### SUMMARY OF THE INVENTION

The present invention is based upon the finding that the ALK inhibition is capable of removing selectively the senescent cells, such finding is supported by the experiments shown in detail in the related experimental section of the present description. In sum, the experiments show that the ALK inhibition by means of Alectinib is capable of removing selectively the senescent cells. Alectinib, is currently used in clinic as antitumour drug in the treatment of the non-small-cell lung carcinoma (NSCLC). Alectinib was selected as senolytic agent after a screening of about 1650 compounds. The experimental data shown in the present patent application show that Alectinib has a senolytic activity comparable to compound ABT-263 (gold standard), in terms of removal of senescent cells (PICS), but it results to be more selective by impacting less seriously on MEF wt (NS). Additional in vitro data on tumour cell lines and human fibroblast demonstrate that Alectinib is a powerful drug capable of eliminating selectively the senescent cells by determining a slowing down of growth in most tumour cells.

Therefore, the present invention firstly relates to ALK tyrosine-kinase receptor inhibitors, in particular Alectinib for use as senolytic drug in a method for the treatment of a tumour or any pathology associated to the cellular senescence. Secondly, it relates to compositions comprising ALK tyrosine-kinase receptor inhibitors, in particular Alectinib, for such use.

Additional advantages, as well as the features and the use modes of the present invention will result evident from the following detailed description of some preferred embodiments.

### DETAILED DESCRIPTION OF THE FIGURES

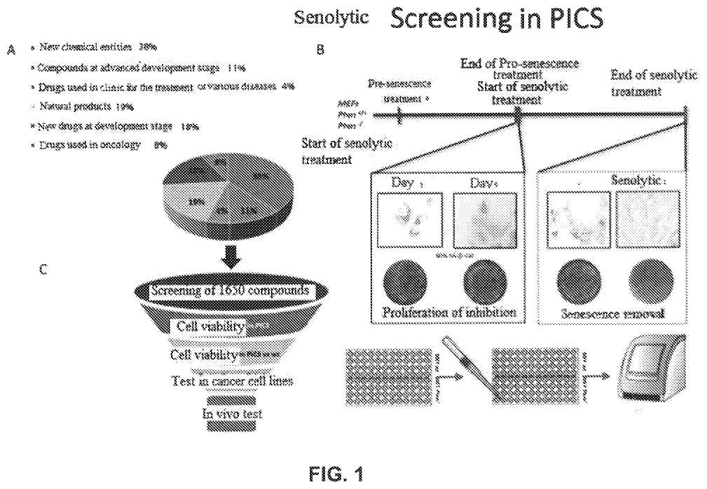

FIG. 1 Platform of senolytic screening in PICS. (A) The circular graph represents the composition, in percentage, of the libraries used in this screening. (B) Screening experimental drawing. MEFs Pten−/− cells were treated for 48 hours with Docetaxel at the concentration of 750 nM with the purpose of inducing senescence. The screening starts when the cell proliferation has stopped and 80% of cells shows a senescent phenotype. The compounds were tested at the single dose of 10 μM. (C) Representation of the different passages thereof the screening is composed. In the first place there is the validation of the compounds in cells wherein Pten a deleted, subsequently the selectivity of these compounds is investigated by inserting even MEF wt, and at last IC50 is evaluated. The compounds satisfying these filters will be then tested on cancer cell lines and at last on in vivo preclinical models.

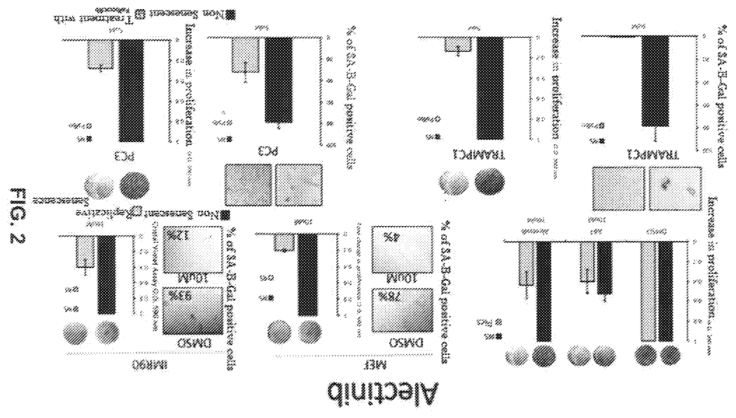

FIG. 2. Identification of Alectinib as senolytic drug. (A) The senolytic screening performed in PICS determined the identification of Alectinib as powerful senolytic drug. This compound shows a strong capability in killing the PICS cells, with respect to ABT-263 considered as positive check. Alectinib shows a marked capability in removing the senescent cells even if the non-senescent, NS, cells are kept vital. Alectinib is capable of removing the MEFs (B) and IMR90 (C) cells in replicative senescence (RS) caused by ageing due to the shortening of telomers, without killing the non-senescent (NS) cells as demonstrated by the assays of Crystal violet and SA-β-Gal. Once having demonstrated the senolytic activity of this compound in primary lines, the activity in cell lines of prostate cancer, such as TrampC1 (D) and PC3 (E), was tested. Our data demonstrate Alectinib's capability of removing the senescent cells in both cell lines.

### DETAILED DESCRIPTION OF THE INVENTION

The present invention is constituted by the following aspects, described in detail hereinafter. The present invention firstly relates to the use of ALK tyrosine-kinase receptor inhibitors as inductors of the selective death of senescent cells. Examples of ALK tyrosine-kinase receptor inhibitors are Ceritinib (CAS 1032900-25-6), Brigantinib (CAS 1197953-54-0), PLB1003, TPX-0005 (CAS#: 2058227-19-1), ASP3026 (CAS No 1097917-15-1), CEP-37440(CAS No 1391712-60-9). According to a preferred embodiment the inhibitor used as senolytic drug is Alectinib. The structure formula of Alectinib is shown hereinafter:

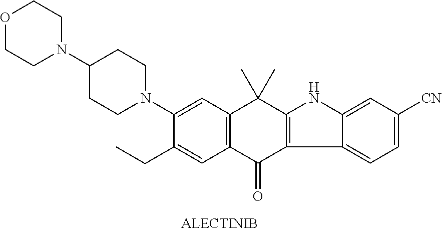 [SMILES: CCc1cc2c(cc1N1CCC(N3CCOCC3)CC1)C(C)(C)c1[nH]c3cc(C#N)ccc3c1C2=O]

In the present description under the term “senolytic drug” a substance is meant capable of removing selectively the senescent cells.

ALK tyrosine-kinase receptor inhibitors, in particular Alectinib, as senolytic drug could be used in a method for the prevention and/or treatment of any pathology associated to the cellular senescence. In the present description under the term “pathology associated to the cellular senescence” any pathological condition is meant due to the accumulation of senescent cells. The selective removal of senescent cells aims at bringing about a therapeutic effect, such as for example in the pathologies related to ageing and in tumour diseases.

Tyrosine-kinase receptor inhibitors could be used as senolytic drugs in the treatment of patients suffering from cancer, preferably in association to one or more chemotherapeutic agents, for example Docetaxel, Palbociclib, Cisplatin and Doxorubicin. Alectinib will be advantageously used in association to one or more chemotherapeutic drugs without senolytic activity.

The treated tumour could be selected for example among prostate, mammary gland, lung, pancreas and colorectal tumour.

The ALK tyrosine-kinase receptor inhibitor, in particular Alectinib as senolytic drug could be used even for the treatment of other pathologies associated to the cellular senescence such as for example osteoarthritis, atherosclerosis, senile dementia, Alzheimer and other geriatric diseases. In the present description, under the expression diseases associated to the cellular senescence, even diseases are meant the treatment thereof involves a cell ageing as side effect, such as for example the treatment of tumour diseases with X rays.

The present invention further relates to a pharmaceutical composition comprising ALK tyrosine-kinase receptor inhibitors, such as for example Alectinib and a carrier and/or a diluent for use as senolytic drug in a method for the treatment of anyone of the herein described conditions to be treated. The composition comprising the ALK tyrosine-kinase receptor inhibitor could be oral, parenteral, rectal, transcutaneous, topic or suitable for other administration route. The compositions for use according to the present invention could be administered by any conventional means available for use together with drugs, or as individual therapeutic agents or in a combination of therapeutic agents. They can be administered alone, but generally administered with a pharmaceutical carrier selected based upon the chosen administration route and the standard pharmaceutical practice. The administered dosage, obviously, will vary depending upon known factors, such as the pharmacodynamic features of the particular agent and upon the administration mode and route thereof; age, health and weight of the recipient; nature and level of symptoms, type of concomitant treatment; frequency of treatment; and wished effect. It can be provided that a daily dosage of active ingredient is about 0.001 until about 1000 milligrams (mg) per kilogramme (kg) of body weight, the preferred dose being 0.1 until about 30 mg/kg. Forms of dosage (compositions suitable for administration) typically include about 1 mg to about 100 mg of active ingredient per dosage unit. In these pharmaceutical compositions, the active ingredient will be usually present in an amount of about 0.1-95% by weight based upon the total weight of the composition.

The ALK tyrosine-kinase receptor inhibitor, such as for example Alectinib can be administered by oral route in solid dosage forms, such as capsules, tablets and powders, or in liquid dosage forms, such as elixirs, syrups and suspensions. It can be administered even by parental route, in sterile liquid dosage forms. Capsules of gelatine include the active ingredient and pulverised carriers, such as lactose, starch, cellulose derivatives, magnesium stearate, stearic acid and the like. Similar diluents can be used to prepare compressed tablets. Both tablets and capsules can be manufactured as prolonged-release products to provide continuous release of medicament for a period of few hours. Compressed tablets can be coated with sugar or coated with film to cover any unpleasant taste and protect the tablet from atmosphere, or coated with enteric coating for the selective disintegration in the gastro-intestinal tract. Liquid dosage forms for oral administration can include dyes and flavours to favour acceptance by the patient. Generally, water, a suitable oil, saline solution, aqueous dextrose (glucose), and solutions of connected sugars and glycols such as propylene glycol or polyethylene glycols are carriers suitable for parental solutions. Solutions for parental administration preferably include a salt soluble in water of the active ingredient, suitable stabilizing agents and, if required, buffering substances. Anti-oxidant agents such as sodium bisulphite, sodium sulphite, or ascorbic acid, alone or combined, are suitable stabilizing agents. Even citric acid and salts thereof and sodium EDTA are used. Moreover, parental solutions can include preservatives, such as benzalkonium chloride, methyl or propyl-paraben, and chlorobutanol. The pharmaceutical compositions according to the present invention apart from Alectinib could include one or more active ingredients, that is pharmacologically active substances, in particular chemotherapeutic agents.

The ALK tyrosine-kinase receptor inhibitor, such as for example Alectinib, could also be used as cosmetic to prevent and/or slow down the cellular senescence not linked to pathological conditions, for example for the improvement and/or prevention of signs of skin ageing such as wrinkles, decrease in softness and/or luminosity of skin and the like.

Herein, the methods are further described for the therapeutic or cosmetic treatment of the above-mentioned pathological conditions comprising a passage of administering ALK receptor inhibitors or compositions including them. The method could be for the treatment of the tumour and it could include a passage preceding the inhibitor administration wherein a patient's sample is analysed to detect the presence or absence of senescent cells.

Herebelow examples are shown having the aim of better illustrating the methods detected in the present description, such examples in no way are to be considered as a limitation of the preceding description and of the following claims.

### Experiments

With the purpose of selecting effectively senolytic drugs the platform was further implemented to identify compounds capable of promoting the senescence previously developed in the inventors' laboratory (see Kalathur, M. et al. A chemogenomic screening identifies CK2 as a target for pro-senescence therapy in PTEN-deficient tumour Nat. Commun. 6, 7227 (2015)).

With the purpose of being able to identify new compounds which could be quickly translated to the clinic, without facing problems of safety, toxicity and pharmacokinetics, we decided to test the senolytic capability of drugs already approved for the use in the oncological field. Our study started with a library of 150 drugs, including inhibitory molecules at the preclinical development stage or currently used in the oncological therapy. Subsequently, our study was extended to a wider range of 1500 compounds including:

(1) new chemical entities;

(2) compounds at advanced development stage;

(3) drugs already used in clinic for the treatment of pathologies of not oncological nature (Neurology, HIV, virology, etc.);

(4) new drugs at development stage (LOPAC) and (5) compounds of natural origin (FIG. 1.A).

The platform which was devised in our laboratory is capable of identifying the senolytic compounds based upon two parameters: (1) the inhibition of the cell viability and (2) the removal of the positive cells to the assay of β-Galactosidase (SA-β-Gal, senescence marker). As positive control of the compounds at the test stage, we included ABT-263, previously identified as senolytic compound 2. At last, the senolytic potentiality of these compounds is tested on murine embryonic fibroblasts (MEF) Pten−/− and WT.

Technically the screening was performed in three passages;
Before treatment, to the culture medium of MEF (DMEM, 10%FBS, 5% P/S) Docetaxel is added, at the concentration of 750 nM for 48 hours. This drug is capable of increasing to 80% the senescence of MEF Pten−/−, without altering the cell cycle of MEF WT (FIG. 1B).
Afterwards, in the cells the culture medium was changed and they were left to grow for 24 hours. The cells were seeded in a 96-well plate at a density of 5000 cells per well. In the more external wells PBS was added, so as to be able to maintain the correct humidity inside the plate.
At last, 24 hours after the seeding of cells, the compounds of our study were added to the cells for 48 hours at the concentration of 10 μM.

The senolytic screening allows to filter the compounds based upon three properties:
Effectiveness: the tested drug in triplicate, at one single concentration (10 μM), in MEF Pten−/− has to be capable of determining a huge decrease in the cell viability.
Specificity: the tested drug in triplicate, at one single concentration (10 μM), in MEF Pten−/− and wt has to be capable of determining a huge decrease in the viability of cells Pten−/− without impacting negatively on MEFs wt.
Dose-Response: the tested drug in duplicate in the assay of dose-response, both in MEF Pten−/− and wt has to allow the calculation of 1050. The data of each plate were normalized on the respective negative control, depending upon the cell population, Ptenfl/fl and Pten−/− (FIG. 10).
As final result of this 1650-compound screening Alectinib (trade name Allcensa) was identified. This drug is ALK inhibitor (Clinical Efficacy of Alectinib in Patients with ALK-Rearranged Non-small Cell Lung Cancer After Ceritinib Failure. Anticancer Res. 37), it is currently used in clinic as antitumour drugs in the treatment of non-small cell lung carcinoma (NSCLC). Our data show that at 10 μM Alectinib has an effectiveness comparable to ABT-263, in terms of removal of senescent cells (PICS), but is results to be more selective by impacting less seriously on MEF wt (NS).

The senolytic activity of Alectinib was validated in the context of the replicative senescence: assay which mimes the natural process of cell ageing due to the shortening of telomers. The MEFs were seeded in 10 cm2-plates (3×105 cells/plate). Every 3 days these cells were detached by means of trypsin, counted with Trypan Blu and plated in the same number. This process was performed for 8 passages before starting the senolytic treatment. At passages 8 and 9 the senescence of these cells was tested by means of the assay of SA-β-Gal (about 78% of senescent cells).

At passage 8, the non-senescent cells (NS) and cells in replicative senescence (RS) were seeded in a 96-well plate (5000 cells/well) and treated at different concentrations of Alectinib. At the concentration of 10 μM, Alectinib is capable of removing the senescent cells without impacting on the cells NS, as demonstrated by the assays of Crystal Violet and SA-β-Gal (FIG. 2B).

This assay was validated even with human fibroblasts, IMR90. Analogously to the protocol used for the MEFs, the IMR90 were seeded in 10 cm2-wide plates (3×105 cells/plate). Every 3 days these cells were detached by means of trypsin, counted with Trypan Blu and plated in the same number. This process was performed for 33 passages before starting the senolytic treatment. At passages 33 and 34 the senescence of these cells was tested by means of the assay of SA-β-Gal (about 78% of senescent cells).

At passage 33 non-senescent (NS) cells and cells in replicative senescence (RS) were seeded in a 96-well plate (5000 cells/well) and treated with different concentrations of Alectinib. At the concentration of 2.5 μM, Alectinib is capable of removing the senescent cells without impacting on the NS cells, as demonstrated by the assays of Crystal Violet and SA-β-Gal (FIG. 2C).

Subsequently we tested the effects of Alectinib on cancer cell lines pre-treated with stimulator of senescence. In the specific case we used TrampC1 and PC3, two cell lines of prostate cancer, respectively murine and human lines. With the purpose of inducing the senescence in these cells, we used Palbociclib, a specific inhibitor of cyclins 4/6 currently used in clinic in the treatment of breast cancer. Once the cells started senescence, Alectinib was added to the culture medium at the concentration of 5 μM for 48 hours. Our data demonstrate that Alectinib is capable of removing successfully the senescent cells as it can be seen from the assays of Crystal Violet and SA-β-Gal both in TrampC1 and PC3 (FIG. 2D E).

In sum, our in vitro data show that Alectinib is a powerful drug capable of removing selectively the senescent cells.

1. A method of preventing and/or treating a pathology associated with cellular senescence to a subject in need thereof comprising administering a therapeutically effective amount of a ALK tyrosine-kinase receptor inhibitor to the subject.

2. The method of claim 1, wherein said pathology is a tumor.

3. The method of claim 2, wherein said tumor is selected from a pancreatic, prostate, lungs, bladder, mammary gland, colorectal, sarcoma, kidney, or thyroid tumor.

4. The method of claim 1, further comprising administering a chemotherapeutic to the subject.

5. The method of claim 4, wherein said chemotherapeutic drug is selected from Docetaxel, Palbociclib, Cisplatin, Doxorubicin or mixtures thereof.

6. A method of reducing the side effects associated with chemotherapy-induced systemic cellular senescence in a subject in need thereof, comprising administering an ALK tyrosine-kinase receptor inhibitor to said subject.

7. The method of claim 1, wherein said pathology is selected from the group consisting of: osteopathies, atherosclerosis, senile dementia, Alzheimer, ageing caused by exposure to UV rays, signs of ageing and/or related pathological and incapacitating conditions.

8. The method of claim 1, wherein said inhibitor is selected from Alectinib, Ceritinib, Brigantinib, PLB1003, TPX-0005, AP26113, ASP3026, or CEP-37440.

9. A method for improving and/or preventing the signs of skin aging in a subject in need thereof, comprising administering a therapeutically effective of a cosmetic composition comprising an ALK tyrosine-kinase receptor inhibitor to the subject.

10. The method of claim 1, wherein the ALK tyrosine-kinase receptor inhibitor is administered as a pharmaceutical composition comprising one or more carriers and/or diluents and/or excipients.

11. The method of claim 10, wherein the pharmaceutical composition further comprises one or more active ingredients.

12. The method of claim 11, wherein said further active ingredients have no senolytic activity.

13. The method of claim 10, wherein said ALK tyrosine-kinase receptor inhibitor is selected from Alectinib, Ceritinib, Brigantinib, PLB1003, TPX-0005, AP26113, ASP3026, or CEP-37440.

14. The method of claim 4, wherein the chemotherapeutic drug is capable of activating senescence.

15. The method of claim 9, wherein the ALK tyrosine-kinase receptor inhibitor is Alectinib.

16. The method of claim 11, wherein the one or more active ingredients is a chemotherapeutic agent.
